(19) United States
(12) Patent Application Publication
(10) Pub. No.: US 2004/0033257 A1
(43) Pub. Date: Feb. 19, 2004

(54) Pharmaceutical formulation in a drug delivery system and process for preparing the same

(75) Inventors: V. S. Iyer, Bangalore (IN); Shivaraj Katageri, Bangalore (IN); Ramachandran Radhakrishnan, Bangalore (IN); Nehru Babu Gaddipati, Somerset, NJ (US)

Correspondence Address: VOLPE AND KOENIG, P.C., UNITED PLAZA, SUITE 1600, 30 SOUTH 17TH STREET, PHILADELPHIA, PA, 19103, US

(73) Assignee: Strides Inc., Somerset, NJ

(21) Appl. No.: 10/222,046

(22) Filed: Aug. 16, 2002

(30) Foreign Application Priority Data
May 30, 2002 (IN) 478/MUM/2002

(51) Int. Cl. A61K 31/473; A61K 9/48; A61K 9/64

(52) U.S. Cl. 424/456; 514/290

(57) ABSTRACT

A process and a pharmaceutical formulation of a water insoluble drug encapsulated in a soft gelatin capsule. The formulation preferably has loratadine as the active ingredient which is formed into a blend with a vehicle which includes a solubilizer, a emulsifier and optionally a viscosity modifying agent. Preferably, the gel encapsulated blend provides a self emulsifying drug delivery system for oral administration of the pharmaceutical formulation.

### FIELD OF THE INVENTION

This invention in general relates to an orally administrable pharmaceutical formulation and a process for preparing the same. More particularly this invention relates to pharmaceutical formulation comprising a water insoluble drug such as loratadine as an active ingredient, encapsulated into gelatin capsules. Preferably, the formulation is in a self-emulsifying drug delivery system.

### BACKGROUND OF THE INVENTION

Loratadine is a water insoluble antihistaminic drug of the formula: ethyl 4-(8-chloro-5,6-dihydro-11-H-benzo[5,6]cyclohepta[1,2-b]pyridin-11-ylidine)-1-piperadine carboxylate. It has a molecular weight of 382.89 g/mol. Loratadine has an antihistaminic effect beginning within 1-3 hours reaching maximum at 8 to 12 hours. Loratadine is indicated for the relief of nasal and non-nasal symptoms of seasonal allergic rhinitis and for the treatment of chronic idiopathic utricaria in patients 2 years of age or older. Loratadine is a long acting tricyclic antihistaminic drug, which has selective peripheral histamine H₁-receptor antagonistic activity. Loratadine is a white to off-white powder which is insoluble in water.

The patient compliance is improved if soft gelatin capsule is used for drug administration, because of its soft, elastic characteristic making it easier to swallow when compared to conventional tablets or hard gelatin capsules.

Furthermore, since the dosage form is generally swallowed, it is unnecessary to flavor or otherwise mask any unpleasant taste of the active pharmaceutical ingredients. Finally, unlike tablets, soft gelatin capsules do not chip or powder.

U.S. Pat. No. 5,827,852 to Russell et al., describes a method for preparing an oral composition of loratadine along with a group of pharmaceutical compounds consisting of pseudoephedrine, phenylephrine, ephedrine, dextromethorphan, noscapine, hydromorphone, glycerol guaiacolate, azatadine, doxylamine, tripelennamine, etc. in tablets, capsules, pills, lozenges, and soft gelatin capsules. Composition of soft gelatin capsules contains PVP, PEG 600, water and ammonium hydroxide. Ibuprofen and pseudoephedrine hydrochloride were present in addition to Loratadine. This patent addresses pharmaceutical compositions suitable for coating and it is not directed at pharmaceutical formulations comprising loratadine as active administered employing soft gelatin capsules.

U.S. Pat. No. 6,086,914 to Weinstein et al., describes a medicinal formulation of loratadine in form of a tablets, capsules, gel caps, powders, or liquids containing methascopolamine nitrate, glycopyrolate, atropine sulfate. This patent addresses a combination therapy, which includes both antihistamine and anti-cholinergic drugs. In this patent components of the formulation are not disclosed.

U.S. Pat. No. 6,217,903 to Skinner et al., sustain release polymer blend pharmaceutical formulation containing loratadine as an antihistaminic agent with other ingredients, polyethylene glycol, stearic acid, colloidal silicon dioxide, magnesium stearate, calcium stearate, waxes, polyvinyl pyrollidone.

U.S. Pat. No. 6,110,498 to Rudnic et al., describes an osmotic drug delivery system containing loratadine by using polyvinyl pyrollidone sodium lauryl sulfate and also some plasticizer like propylene glycol, triethyl citrate, and vegetable oil. The invention disclosed in this patent does not address soft gelatin capsules. This patent provides an osmotic drug delivery system, preferably in the form of a tablet comprising various components like polymers sprayed on tablets to give 2-15% coating weight, wicking agents, non-swelling solubilizing agents and lubricating agents.

U.S. Pat. No. 5,385,941 to Fawzi et al., describes a novel pharmaceutical composition of loratadine are comprising of salt or ion pair formation of non-steroidal anti-inflammatory drug and an anti-histamine or other decongestant dosage form such as capsules, tablets, elixirs and ointments. This disclosure relates to dry mix formulations for biophosphonic acids with lactose. The use of soft gelatin capsules for administering pharmaceutical active comprising loratadine are not disclosed.

### SUMMARY OF THE INVENTION

A pharmaceutical delivery device is provided comprising a blend of a water insoluble antihistamine drug and a vehicle which is encapsulated in a gelatin capsule. Preferably, the active drug ingredient is loratadine which is blended with a vehicle to provide a self-emulsifying drug delivery system for administering orally the said pharmaceutical formulation.

It is another aspect of the invention to employ a soft gelatin drug delivery system for oral administration of the pharmaceutical formulation. For the purpose of attaining good dissolution properties for the dosage form, several surfactants and solubility enhancers were used, which provides a self-emulsifying drug delivery system, which help to enhance bioavailability.

The objective of the present invention is to provide a cost effective, rapidly released, uncoated, taste-masked, non-sedating antihistamine dosage form. It is known that the bioavailability of drugs in solubilized form is higher than that in solid dosage forms. The present invention offers a soft gelatin capsule containing solubilized loratadine, where after the rupturing of the gelatin shell (normally 2 to 5 minutes) in body fluids, the drug is released.

In accordance with yet another aspect there are provided soft gelatin capsules of a pharmaceutical formulations comprising of loratadine, diethylene glycol monoethyl ether, medium chain triglycerides of coconut oil and caprylo capryl macrogol glycerides.

In accordance with still another aspect of the invention, there are provided soft gelatin capsule of a pharmaceutical formulation comprising of loratadine, diethylene glycol monoethyl ether, polyethylene glycol (PEG 400), caprylo capryl macrogol glycerides and PVP K 30.

In accordance with another aspect there are provided methods of making a pharmaceutical formulation comprising Loratadine as the active pharmaceutical ingredient, diethylene glycol monoethyl ether, and caprylo capryl macrogol glycerides.

It is also an aspect of the invention to provide for methods of making soft gelatin capsule of a pharmaceutical formulation comprising of loratadine, absolute alcohol, glyceryle triacetate and of caprylo capryl macrogol glycerides.

In still another aspect there are provided methods of making soft gelatin capsule of a pharmaceutical formulation comprising loratadine, absolute alcohol, glyceryle triacetate and polyoxyl 35 castor oil.

Also it is an aspect of the invention to provide for methods of making soft gelatin capsule of a pharmaceutical formulation comprising loratadine, absolute alcohol, polyethylene glycol esters of tetrahydro furfuryl alcohol and caprylo capryl macrogol glycerides.

### DETAILED DESCRIPTION OF THE PREFERRED EMBODIMENT

The present invention aims at developing a pharmaceutically active blend of a water insoluble drug, in particular, loratadine, and a vehicle, enclosed in soft gelatin capsules. It is desirable to use as small amount of the blend as practical. Usual recommended strength of loratadine is 10 mg/dosage unit. Products currently available in market under the trademark Claritin tablets and Claritin Reditabs contain 10 mg of loratadine, an antihistamine, to be administered orally. Human histamine skin wheal studies following single and repeated doses of loratadine have shown that the drug exhibits an antihistamine effect beginning within 1 to 3 hours, reaching a maximum at 8 to 12 hours (Physician's Desk Reference, 56^{th} ed., 2002, p. 3100). Additional strengths of loratadine tablets are available up to a maximum of 40 mg/dosage unit. Similarly, in the case of soft gelatin capsules, the strength may vary from 10 mg to 40 mg/dosage unit with the other excipients increasing proportionately. The size and shape of capsules may vary depending on the loratadine content, and may be oval, oblong or round in shape.

Loratadine is solubilized in a vehicle. A vehicle can be composed of various excipients, and may be either solid or liquid. Within the preferred embodiment of this invention, the vehicle is liquid.

The physical characteristic of the blend of loratadine and the vehicle may vary depending on the excipients and the preparatory methods. The solubilized loratadine in the vehicle may be a homogeneous mixture, such as a solution, or may be a heterogeneous mixture, such as a suspension. Heterogeneous blend may have particles with sufficient size that the blend exhibits the Tyndall Effect, or it may be a microsuspension, where the discontinuous phase does exist, but is of such small particle size to be imperceptible to a particle size measuring apparatus.

The liquid vehicle is comprised of a solvent liquid that solubilizes (i.e., either dissolves or suspends) the active ingredient, a blend of surfactants, and optionally a viscosity modifier. The solvent liquid may be composed of either one, or several different solvents. The vehicle may be comprised of water immiscible liquids (such as oils, hydrocarbons, glycerides), water miscible liquids (such as PEG, alcohols, ethers), or a combination of both.

Since loratadine is water insoluble, it was necessary to utilize organic solvents that solubilize the drug. Solvents like PEG 400, 1,2-propylene glycol, diethylene glycol monoethyl ether, glyceryl triacetate, absolute alcohol and vegetable oils were used for solubility studies.

Transcutol® P solubilizer is used to dissolve loratadine. Transcutol P is purified diethylene glycol monoethyl ether that acts as a powerful solubilizer for several poorly soluble drugs. It is soluble in water, ethanol, hexylene glycol and propylene glycol, and partially soluble in vegetable oils. It also acts as a co-surfactant in the formulation.

Miglyol is medium chain triglyceride mixture obtained from coconut oil. It is used as the oil phase for the formulation of the self-emulsifying drug delivery system.

Liquid polyethylene glycols are used as water miscible solvents for the contents of soft gelatin capsules. PEG 400 is used as a solvent in this formulation, though polyethylene glycol liquid grades of PEG 200 to 600 may be utilized as well. Polyethylene glycols are hydrophilic solvents that occur as clear, colorless or slightly yellow colored viscous solutions, have a slight but characteristic odor, and a bitter, burning taste.

For the purpose of attaining good dissolution properties for the dosage form, several surfactants, emulsifiers and solubility enhancers were used. These include polyglycolized glycerides, fatty acid esters of vegetable oils and hydrogenated oils, Tween® and Span® agents. Polyvinyl Pyrrolidone (PVP) K30 was used as a solubility enhancer as it is known to form a complex with insoluble molecules and those are difficult to solubilize and hence to give good dissolution and enhanced bioavailability.

Labrasol® emulsifier is caprylocaproyl macrogol-8 glycerides. Labrasol is a non-ionic emulsifier that can form a self-emulsifying drug delivery system. Labrasol improves the in-vitro dissolution properties and oral absorption of poorly soluble compounds because of their self-emulsifying properties. Mixed with an oil, they have the ability to spontaneously form an emulsion of fine droplets of uniform size distribution when comes in contact with aqueous media (in vitro) or physiological fluid (in vivo). The oil in water emulsion gives a large surface area for absorption of the drug. Bioavailability of drugs dissolved in Labrasol in an emulsion pre-concentrate may be increased to 3-4 fold compared to systems with the drug in a lipophilic vehicle or in liquid PEG systems. HLB value of Labrasol is 14. Another surfactant used in these formulation, Polyoxyl 35 castor oil has a HLB value of 12-14. Absorption of drugs formulated in Labrasol are generally not affected by the presence or absence of food. After administration and spontaneous emulsification in the gastrointestinal fluids, these products follow the fate of lipid type products.

The vehicle may optionally contain a viscosity modifier. Polyvinyl pyrrolidone, or povidone, is a fine, white to creamy white, odorless or almost odorless, hygroscopic powder. It has a molecular weight of approximately 2,500 to 400,000 daltons. Povidone used in present formulations is Kollidon® from BASF, however, other brands of polyvinyl pyrrolidone may be used.

The following examples illustrate preferred embodiments of pharmaceutical compositions comprising loratadine as active ingredient.

TABLE 1

| | 1 | 2 | 3 | 4 | 5 | 6 |
|---|---|---|---|---|---|---|
| Ingredients | Quantity per capsule (mg) | | | | | |
| Loratadine | 10 | 10 | 10 | 10 | 10 | 10 |
| Transcutol P | 50 | 50 | 90 | | | |
| Absolute alcohol | | | | 20 | 20 | 20 |
| Miglyol | 40 | | | | | |
| Glyceryl triacetate | | | | 80 | 80 | |
| Polyethylene glycol esters | | | | | | 80 |
| of tetrahydro furfuryl alcohol | | | | | | |
| PEG 400 | | 35 | | | | |
| Labrasol | 25 | 25 | 25 | 25 | | 25 |
| Polyoxyl 35 castor oil | | | | | 25 | |
| Kollidon | | 5 | | | | |

In general, gelatin capsule formulations for soft gelatin capsules comprise raw gelatin and one or more plasticizers added to adjust the hardness of the capsule. Typical plasticizers include glycerin, sorbitol and Anidrisorb 85/70. A preferred plasticizer is Anidrisorb 85/70, an aqueous solution of D-sorbitol and sorbitans. One preferred gelatin formulation for the soft gelatin capsules used in accordance with preferred embodiments includes gelatin in the range of about 40% to 48% and a plasticizer ranging in amount from about 16% to 35%. Another preferred plasticizer is sorbitol, a non-crystallizing sorbitol solution. When either a 70%, non-crystallizing sorbitol solution or Anidrisorb 85/70 are used alone, the amount of plasticizer used preferably ranges from about 16% to 35%. Capsule formulations can also include other suitable additives such as anti-oxidants, amino acids and coloring agents, which impart specific characteristics including capsule aesthetics. FD&C dyes and D&C dyes are examples of pharmaceutically acceptable coloring agents that may be used in preferred embodiments.

The following examples illustrate preferred embodiments of several soft-gelatin-shell loratadine formulations. These examples illustrate particular embodiments of the invention and are not intended to limit the scope of the invention in any way.

TABLE 2

| | 7 | 8 | 9 | 10 |
|---|---|---|---|---|
| Ingredient | Weight percent (min-max) | | | |
| Gelatin | 38.0 | 38.0 | 38.0 | 38.0 |
| | 46.0 | 46.0 | 46.0 | 46.0 |
| Sorbitol Solution | 14.0 | 14.0 | 14.0 | 14.0 |
| | 25.0 | 25.0 | 25.0 | 25.0 |
| Glycine | 0.2-0.6 | 0.2-0.6 | 0.2-0.6 | 0.2-0.6 |
| Butylated Hydroxy | 0.02 | 0.02 | 0.02 | 0.02 |
| Anisole | 0.03 | 0.03 | 0.03 | 0.03 |
| Butylated | | 0.02 | | 0.02 |
| HydroxyToluene | | 0.03 | | 0.03 |
| Citric Acid | | | 0.42 | 0.42 |
| | | | 0.46 | 0.46 |
| Purified water | 40.5 | 40.5 | 40.5 | 40.5 |
| | 45.5 | 45.5 | 45.5 | 45.5 |

Manufacturing of loratadine capsules is carried out under cGMP conditions. The general method of manufacturing of loratadine blend involves solubilization of loratadine in Transcutol or in absolute alcohol in a suitable stainless steel (SS) mixing vessel and adding other ingredients into this blend in a suitable SS mixing vessel (preferably planetary mixer), with stirring at preferably 20 rpm at ambient conditions, to obtain a clear free flowing solution. The pH of obtained blend may vary from 5.0 to 8.5. Gelatin paste preparation is carried out in a melter. The gelatin paste preparation is done by heating the gelatin with plasticizer and purified water with continuous stirring. During gelatin paste preparation, vacuum is applied to remove extra amounts of water added and to get a gelatin ribbon free from air bubbles. Colorants may be optionally added and mixed further in SS tank at 60±5° C. for 1 to 2 hours to get a uniform color distribution. The blend of loratadine and gelatin paste as obtained above are further taken for encapsulation. Manufacturing of soft gelatin capsules is carried out using rotary die process. However other processes like plate process, accogel process and bubble method may used. The shape of capsule may be oval, round or oblong, most preferably oval shaped. Encapsulation process is carried out at temperature below 30° C. and relative humidity below 25%.

The stated prior art shows a need for improvements in the area of solubilization of the loratadine. The dissolution profile of loratadine soft gelatin capsules in different media was comparable with loratadine rapidly-disintegrating tablets. The details are as follows:

| | | % of drug released after | | | |
|---|---|---|---|---|---|
| Product | Media | 5 min. | 10 min. | 20 min. | 30 min |
| Loratadine soft gelatin | 0.1 M | 94.4 | 99.8 | 103.0 | 103.6 |
| capsules 10 mg | HCl | | | | |
| Loratadine rapidly | 0.1 M | 98.7 | 98.6 | 98.2 | 100.4 |
| disintegrating tablets | HCl | | | | |
| 10 mg | | | | | |
| Loratadine soft gelatin | pH 7.5 | 20.4 | 22.3 | 23.2 | 23.8 |
| capsules 10 mg | | | | | |
| Loratadine rapidly | pH 7.5 | 23.4 | 24.6 | 25.6 | 27.2 |
| disintegrating tablets | | | | | |
| 10 mg | | | | | |
| Loratadine soft gelatin | pH 6.5 | 32.8 | 42.0 | 45.0 | 50.0 |
| capsules 10 mg | | | | | |
| Loratadine rapidly | pH 6.5 | 33.0 | 39.5 | 39.5 | 50.0 |
| disintegrating tablets | | | | | |
| 10 mg | | | | | |
| Loratadine soft gelatin | 0.3% | 83.6 | 92.0 | 98.0 | 99.0 |
| capsules 10 mg | SLS | | | | |
| Loratadine rapidly | 0.3% | 95.4 | 97.5 | 98.0 | 97.6 |
| disintegrating tablets | SLS | | | | |
| 10 mg | | | | | |

Certain modifications and improvements of the disclosed invention will occur to those skilled in the art without departing from the scope of the invention.

We claim:

1. A pharmaceutical delivery device comprising:
a blend of a water insoluble antihistaminic drug and a vehicle; and
a gelatin capsule.

2. Pharmaceutical delivery device according to claim 1, wherein said gelatin capsule is a soft gelatin capsule.

3. Pharmaceutical delivery device according to claim 2, wherein said blend is a solution.

4. Pharmaceutical delivery device according to claim 2, wherein said blend is a suspension.

5. Pharmaceutical delivery device according to claim 2, wherein said drug is loratadine.

6. Pharmaceutical delivery device according to claim 5, wherein said blend is self-emulsifying.

7. Pharmaceutical delivery device according to claim 6, wherein said vehicle is comprised of at least one water-miscible solubilizer, at least one water-immiscible solubilizer, and at least one emulsifier.

8. Pharmaceutical delivery device according to claim 7, wherein said water-immiscible solubilizer is an oil.

9. Pharmaceutical delivery device according to claim 8, wherein said oil is a vegetable oil.

10. Pharmaceutical delivery device according to claim 7, wherein said water-immiscible solubilizer is medium chain triglycerides.

11. Pharmaceutical delivery device according to claim 6, wherein said vehicle is comprised of a solubilizer and an emulsifier.

12. Pharmaceutical delivery device according to claim 11, herein solubilizer is water miscible.

13. Pharmaceutical delivery device according to claim 11, wherein said solubilizer is selected from a group consisting of diethylene glycol monoethyl ether, absolute alcohol, glyceryl triacetate, polyethylene glycol esters of tetrahydrofurfuryl alcohol, polyethylene glycol, propylene glycol, and medium chain triglycerides.

14. Pharmaceutical delivery device according to claim 13, wherein said emulsifier is caprylocapryl macrogol glycerides.

15. Pharmaceutical delivery device according to claim 11, wherein said emulsifier is selected from a group consisting of reaction products of natural or hydrogenated vegetable oils with ethylene glycol, polyoxy ethylene fatty acid esters, propylene glycol mono and di-fatty acid esters, trans-esterification products of natural vegetable oils and poly alkylene polyols, monoglycerides, diglycerides, esterification products of caprylic/capric acids with glycerol, sorbitan fatty acid esters, pentaerythrol fatty acid esters, polyalkylene glycerol esters, monoglycerides-glyceryl monosterate, glyceryl monopalmitate, glyceryl monooleate, acetylated monoglycerides, glyceryl triacetate, sterols, and derivatives thereof.

16. Pharmaceutical delivery device according to claim 14, wherein said solubilizer is diethylene glycol monoethyl ether.

17. Pharmaceutical delivery device according to claim 14, wherein the emulsifier has an HLB value between about 7 and about 20.

18. Pharmaceutical delivery device according to claim 14, wherein the emulsifier has an HLB value between about 12 and about 14.

19. Pharmaceutical delivery device according to claim 11, wherein said emulsifier comprises a polyglycolized glyceride.

20. Pharmaceutical delivery device according to claim 19, wherein said polyglycolized glyceride is caprylocaproyl macrogol glycerides.

21. Pharmaceutical formulation according to claim 11, wherein said vehicle further comprises a viscosity modifing agent.

22. Pharmaceutical formulation according to claim 21, wherein the viscosity modifying agent comprises povidone.

23. Pharmaceutical formulation according to claim 22, wherein the povidone has a molecular weight between about 2,500 and 400,000 g/mol.

24. Pharmaceutical formulation according to claim 11, wherein said solubilizer is diethylene glycol monoethyl ether.

25. Pharmaceutical formulation according to claim 24, wherein said emulsifier comprises a polyglycolized glyceride.

26. Pharmaceutical formulation according to claim 11, wherein said solubilizer is glyceryl triacetate.

27. Pharmaceutical formulation according to claim 26, wherein said emulsifier comprises a polyglycolized glyceride.

28. A method of manufacturing a pharmaceutical delivery device comprising:
blending a water insoluble antihistaminic drug and a vehicle to produce a blend; and
encapsulating the blend in a gelatin capsule.

29. The method according to claim 28 wherein said drug is loratadine which is blended with a vehicle selected to produce a self-emulsifying blend.

30. The method according to claim 29 wherein said vehicle is selected such that it includes at least one water-miscible solubilizer, at least one water-immiscible solubilizer, and at least one emulsifier.

31. The method according to claim 29 wherein said vehicle is selected such that it includes a solubilizer and an emulsifier.
